SEQUENCE LISTING (32 sequences)

SEQ ID NO: 1 - DNA, 20 bp, Artificial Sequence
  An artificially synthesized primer sequence
tgtatgaagccatcttcacg

SEQ ID NO: 2 - DNA, 20 bp, Artificial Sequence
  an artificially synthesized primer sequence
ggcattgaacttcatagcga

SEQ ID NO: 3 - DNA, 20 bp, Artificial Sequence
  An artificially synthesized primer sequence
tcaaaccctttaccacggtt

SEQ ID NO: 4 - DNA, 20 bp, Artificial Sequence
  An artificially synthesized primer sequence
caggctctactttgatcgca

SEQ ID NO: 5 - DNA, 21 bp, Artificial Sequence
  An artificially synthesized primer sequence
ggagccatggattgcacattt

SEQ ID NO: 6 - DNA, 21 bp, Artificial Sequence
  An artificially synthesized primer sequence
tccttccgaaggtctctcctc

SEQ ID NO: 7 - DNA, 24 bp, Artificial Sequence
  An artificially synthesized primer sequence
agagccatcagacagccccgagat

SEQ ID NO: 8 - DNA, 24 bp, Artificial Sequence
  An artificially synthesized primer sequence
tgacgagtagaggggcatgtggag

SEQ ID NO: 9 - DNA, 20 bp, Artificial Sequence
  An artificially synthesized primer sequence
aggtgacgtgctagtgatgg

SEQ ID NO: 10 - DNA, 20 bp, Artificial Sequence
  An artificially synthesized primer sequence
ctccgtggtggatacgttct

SEQ ID NO: 11 - DNA, 20 bp, Artificial Sequence
  An artificially synthesized primer sequence
gtgcggttcttcctgcaact

SEQ ID NO: 12 - DNA, 20 bp, Artificial Sequence
  An artificially synthesized primer sequence
aacagcaggaagggcttgtg

SEQ ID NO: 13 - DNA, 21 bp, Artificial Sequence
  An artificially synthesized primer sequence
atggcagtccggagaataccc

SEQ ID NO: 14 - DNA, 22 bp, Artificial Sequence
  An artificially synthesized primer sequence
cctcataacgctggcttcgagt

SEQ ID NO: 15 - DNA, 20 bp, Artificial Sequence
  An artificially synthesized primer sequence
aggtcattcctagccgatac

SEQ ID NO: 16 - DNA, 20 bp, Artificial Sequence
  An artificially synthesized primer sequence
tacatcctctggcttgctac

SEQ ID NO: 17 - DNA, 20 bp, Artificial Sequence
  An artificially synthesized primer sequence
atcaggctgcttatggatga

SEQ ID NO: 18 - DNA, 20 bp, Artificial Sequence
  An artificially synthesized primer sequence
ttcactgacgtgtttgcttg

SEQ ID NO: 19 - DNA, 20 bp, Artificial Sequence
  An artificially synthesized primer sequence
ggtgggccacaaattacgtg

SEQ ID NO: 20 - DNA, 20 bp, Artificial Sequence
  An artificially synthesized primer sequence
cagcatctccatggcgtagt

SEQ ID NO: 21 - DNA, 20 bp, Artificial Sequence
  An artificially synthesized primer sequence
tccgaggtgtcatcctcact

SEQ ID NO: 22 - DNA, 20 bp, Artificial Sequence
  An artificially synthesized primer sequence
tgcacagccttccagtactc

SEQ ID NO: 23 - DNA, 19 bp, Artificial Sequence
  An artificially synthesized primer sequence
tgggcccagcttcttagcc

SEQ ID NO: 24 - DNA, 21 bp, Artificial Sequence
  An artificially synthesized primer sequence
ggaacagcgcagtaccgtaga

SEQ ID NO: 25 - DNA, 20 bp, Artificial Sequence
  An artificially synthesized primer sequence
tctcccaatgttgcccgaaa

SEQ ID NO: 26 - DNA, 20 bp, Artificial Sequence
  An artificially synthesized primer sequence
gggaggtcgtctgtcatgag

SEQ ID NO: 27 - DNA, 21 bp, Artificial Sequence
  An artificially synthesized primer sequence
ggaaacttgtgcaaggttgga

SEQ ID NO: 28 - DNA, 20 bp, Artificial Sequence
  An artificially synthesized primer sequence
ggtcacccttaggaccaaga

SEQ ID NO: 29 - DNA, 20 bp, Artificial Sequence
  An artificially synthesized primer sequence
atggatctcaaagacaacca

SEQ ID NO: 30 - DNA, 20 bp, Artificial Sequence
  An artificially synthesized primer sequence
tcctggtatgaaatggcaaa

SEQ ID NO: 31 - DNA, 24 bp, Artificial Sequence
  An artificially synthesized primer sequence
gtcccagtatgcctctggtcgtac

SEQ ID NO: 32 - DNA, 24 bp, Artificial Sequence
  An artificially synthesized primer sequence
ccacgctcggtcaggatcttcatg

(19) United States
(12) Patent Application Publication
(10) Pub. No.: US 2010/0222259 A1
(43) Pub. Date: Sep. 2, 2010

(54) Compositions for enhancing the production of PPAR and/or PPAR-associated factors

(76) Inventors: Hidekazu Arai, Tokushima (JP); Eiji Takeda, Tokushima (JP); Hajime Sasaki, Kanagawa (JP)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/631,745

(22) PCT Filed: Apr. 1, 2005
(86) PCT No.: PCT/JP05/06461

§ 371 Date: Feb. 21, 2008

(30) Foreign Application Priority Data
Jul. 9, 2004 (JP) 2004-203539
Oct. 1, 2004 (JP) 2004-290843

(51) Int. Cl. A61K 38/16; A61P 3/10; A61P 3/04

(52) U.S. Cl. 514/12

(57) ABSTRACT

The present inventors focused on certain nutritional compositions known to have activity of controlling blood glucose levels. These foods were administered to rats for long periods, and real-time PCR was used to analyze the expression of genes associated with lipid metabolism in the liver and adipose tissues. As a result, the present inventors found that the expression of the PPARα gene is enhanced by these foods, and that this is accompanied by suppressed expression of fatty acid synthase and enhanced expression of a group of PPARα target genes associated with fatty acid metabolism. The present inventors also confirmed the effect of these foods in enhancing the expression of PPARγ and adiponectin, and discovered that these foods have the activity of enhancing the production of PPAR and PPAR-associated factors.

### TECHNICAL FIELD

The present invention relates to compositions with the activity of enhancing the production of PPARs and/or factors associated with PPARs, and foods supplemented with effective amounts of such compositions.

### BACKGROUND ART

Obesity prevention and treatment is very important in maintaining and promoting health. Obesity leads to type II diabetes, hypertension, hyperlipidemia, and such. These diseases are also the underlying diseases causing cerebral stroke, ischemic heart diseases, and such. The current understanding is that these diseases are a series of metabolically abnormal conditions based on insulin resistance caused by obesity. Recent molecular biological research has revealed the existence of various factors involved in obesity and insulin resistance.

Adiponectin is one of the substances receiving attention as an above-mentioned factor. Adiponectin (Acrp30/AdipoQ/GBP28) is an adipocytokine that improves insulin resistance. Adiponectin was identified as a gene expressed most abundantly in human adipose tissue (Non-Patent Document 1). Adiponectin concentration in the blood is reduced in obesity, diabetes, and ischemic heart diseases, and adiponectin has been confirmed to have anti-diabetic and anti-arteriosclerotic activities (Non-Patent Documents 2-6). Hypoadiponectinemia caused by obesity and fat accumulation is thought to lead to insulin resistance syndromes such as diabetes and hyperlipidemia, systemic metabolic syndromes, arteriosclerosis, and such (Non-Patent Document 7).

Along with adiponectin, PPARs are also factors involved in obesity and insulin resistance. Peroxisome proliferator-activated receptors (PPARs) are transcription factors belonging to the nuclear receptor superfamily, and mammals have α, γ, and δ subtypes. PPARs form heterodimers with retinoid X receptors (RXR), and induce expression of target genes with a PPAR-responsive element (PPRE) in their promoter region in a ligand-dependent manner.

Subtype PPARγ is known to regulate adipocyte differentiation and hypertrophy. Studies using heterozygous PPARγ knockout mice have shown that PPARγ mediates obesity and insulin resistance. Suppression of adipocyte hypertrophy by suppressing the endogenous activity of PPARγ is considered to be useful as an anti-obesity and anti-diabetic therapy (Non-Patent Document 7). On the other hand, thiazoline derivatives, which are insulin sensitizers, are known to be PPARγ agonists. The small adipocyte hypothesis has been proposed as an explanation for this seemingly contradictory situation (Non-Patent Document 7). This hypothesis is that adipocytes include small adipocytes and large adipocytes, and that these respective adipocytes show opposite effects on insulin resistance. PPARγ holds the key to adipocyte differentiation and hypertrophy. According to this hypothesis, PPARγ stores fat in adipocytes during a high-fat diet, increasing the number of large adipocytes. Hypertrophic adipocytes secrete adipocytokines such as TNFα and resistin, which worsen insulin resistance. On the other hand, since PPARγ causes preadipocytes to differentiate into small adipocytes that secrete high levels of adipocytokines such as leptin and adiponectin, which improve insulin resistance, insulin resistance is improved by PPARγ agonists.

PPARα is secreted at particularly high levels in the liver, and its main target is a group of genes involved in the use of fatty acids (Non-Patent Document 8).

Recently, a close relationship between these factors has been reported. PPRE exists in the adiponectin promoter region, and binding of PPARγ was found to induce adiponectin expression (Non-Patent Document 9). There are also reports that adiponectin acts on the liver to induce PPARα expression, and activates the endogenous ligand activity of PPARα (Non-Patent Documents 10 and 11).

Since PPARs and adiponectin function as insulin sensitizers, as described above, they may be effective for preventing and treating obesity and diabetes. Since diabetes prevention and treatment is based on suitable dietary management, if foods that enable activation or enhanced production of PPARs or adiponectin can be developed, such foods may become an effective means for diabetes prevention and treatment. Furthermore, these foods are expected to be effective against other diseases based on insulin resistance. However, there are no specific reports of examples of foods that enhance adiponectin production.
[Non-Patent Document 1] Maeda, K., Okubo, K., Shimomura, I., et al.: cDNA cloning and expression of a novel adipose specific collagen-like factor, apM1 (Adipose Most Abundant Gene transcript 1). Biochem. Biophys. Res. Commun. 1996; 221:286-289.
[Non-Patent Document 2] Hotta, K., Funahashi, T., Arita, Y., et al.: Plasma concentrations of a novel, adipose-specific protein, adiponectin, in type 2 diabetic patients. Arterioscler. Thromb. Vasc. Biol. 2000; 20: 1595-1599.
[Non-Patent Document 3] Ouchi, N., Kihara, S., Arita, Y., et al.: Novel modulator for endothelial adhesion molecules: adipocyte-derived plasma protein adiponectin. Circulation 1999; 100: 2473-2476.
[Non-Patent Document 4] Kondo, H., Shimomura, I., Matsukawa, Y., et al.: Association of adiponectin/ACRP 30/AdipoQ mutation with type 2 diabetes mellitus. A candidate gene for the insulin resistance syndrome. Diabetes 2002; 51: 2325-2328.
[Non-Patent Document 5] Maeda, N., Shimomura, I., Kishida, K., et al.: Diet-induced insulin resistance in mice lacking adiponectin/ACRP 30. Nature Medicine 2002; 8: 731-737.
[Non-Patent Document 6] Matsuda, M., Shimomura, I., Sata, M., et al.: Role of adiponectin n preventing vascular stenosis—the missing link of adipo-vascular axis—. J. Biol. Chem. 2002; 277: 37487-37491.
[Non-Patent Document 7] Kadowaki, T.: Molecular Mechanism of Insulin Resistance by Adipocytes. The 124th Japanese Association of Medical Sciences Symposium Records, “Science of Obesity”; p 110-121 (2003).
[Non-Patent Document 8] Frohnert, B. I., Hui, T. Y. and Bernlohr, D. A.: Identification of a functional peroxisome proliferator-responsive element in the murine fatty acid transport protein gene. J. Biol. Chem. Vol. 274 No. 7, 3970-3977 (1999).
[Non-Patent Document 9] Iwaki, M. et al.: Induction of adiponectin, a fat-derived antidiabetic and antiatherogenic factor, by nuclear receptors. Diabetes 52, 1655-1663 (2003).
[Non-Patent Document 10] Yamauchi, T. et al.: Cloning of adiponectin receptors that mediate antidiabetic metabolic effects. Nature 423, 762-769 (2003).
[Non-Patent Document 11] Yamauchi, T. et al.: The fat-derived hormone adiponectin reverses insulin resistance associated with both lipoatrophy and obesity. Nature Medicine Vol. 7 No. 8, 941-946 (2001)
[Patent Document 1] WO03/022288

### DISCLOSURE OF THE INVENTION

### Problems to be Solved by the Invention

A problem to be solved by the present invention is the provision of foods with the activity of enhancing the production of PPARs or PPAR-associated factors.

### Means to Solve the Problems

To solve the above-mentioned problem the present inventors conducted dedicated studies to search for foods with the activity of enhancing the production of PPARs and PPAR-associated factors, with a focus on certain nutritional compositions (Patent Document 1). The major sources of carbohydrates in the foods mentioned above are slowly absorbed carbohydrates, and their characteristic fatty acid composition is rich in oleic acid and α-linolenic acid. These foods are known to have effect of controlling blood glucose levels (Patent Document 1), but thus far the mechanism of their action had not been elucidated. Therefore, the present inventors administered the foods to rats for long periods, and used real-time PCR to analyze the gene expression associated with lipid metabolism in the liver and adipose tissues. As a result, the present inventors found that these foods enhanced expression of the PPARα gene, which in turn suppresses expression of fatty acid synthase, and enhances expression of a group of PPARα target genes associated with fatty acid metabolism. The present inventors further confirmed the effect of the foods in enhancing expression of PPARγ and adiponectin, discovering that the above-mentioned foods have the activity of enhancing the production of PPARs and PPAR-associated factors.

The present invention thus relates to foods with the activity of enhancing the production of PPARs and PPAR-associated factors, and more specifically, it provides the following inventions:

[1] a nutritional composition for enhancing the production of a PPAR and/or PPAR-associated factor, wherein the composition comprises proteins, fats, and carbohydrates such that proteins account for 10% to 25% of its energy, fats account for 10% to 35% of its energy, and carbohydrates account for 40% to 60% of its energy, and wherein oleate esters account for 60% to 90% of the energy in the fats, and palatinose and/or trehalose account for 60% to 100% of the energy in the carbohydrates;
[2] the composition of [1], wherein the PPAR is a PPARα and/or a PPARγ;
[3] the composition of [1], wherein the PPAR is a PPARγ;
[4] the composition of any of [1] to [3], wherein the composition for enhancing the production of the PPAR-associated factor is adiponectin;
[5] the composition of any of [1] to [4], which comprises at least one fat selected from a milk phospholipid, a soybean lecithin, a high oleic sunflower oil, and a perilla oil; and
[6] a food for treating a patient with diabetes, treating a patient with impaired glucose tolerance, or preventing obesity, which comprises the composition of any of [1] to [5].

### EFFECTS OF THE INVENTION

The nutritional compositions of the present invention have the activity of enhancing the production of PPARs and adiponectin, which act as insulin sensitizers; they are therefore useful as oral and tube nutritional agents, therapeutic foods, foods for patients at home, or foods with health claims, which are used for preventing and treating obesity and diabetes. The compositions are also expected to be effective against other insulin resistance-based diseases, such as hyperlipidemia and hypertension.

### BRIEF DESCRIPTION OF THE DRAWINGS

FIG. 1 shows the relative expression levels of PPARα, PPARγ, and SREBP-1c in each tissue (A: liver; B: adipose tissue) of rats (test sample group, MBC group, and MF group).

FIG. 2 shows the relative expression levels of genes associated with lipid metabolism (A: lipase; B: fatty acid transport protein; and C: fatty acid synthesis) in the livers of rats (test sample group, MBC group, and MF group).

FIG. 3 shows the relative expression levels of genes associated with fatty acid β-oxidation in the liver of rats (test sample group, MBC group, and MF group) (A: β-oxidation within the peroxisome; B: β-oxidation within the mitochondria).

FIG. 4 shows the relative expression levels of adipocytokines (A: Acrp30 (adiponectin); B: TNFα) in the adipose tissue of rats (test sample group, MBC group, and MF group).

FIG. 5 shows the relative expression levels of UCP2 in each tissue (A: liver; B: adipose tissue) of rats (test sample group, MBC group, and MF group).

FIG. 6 shows the changes in (A) RQ, (B) the amount of carbohydrates burned, and (C) amount of fat burned over time after intake of the test sample and the control. The results are represented as mean±standard error.

FIG. 7 shows the changes in (A) plasma glucose level, (B) serum insulin concentration, and (C) free fatty acid concentration in serum, over time after intake of the test sample and the control. The results are represented as mean±standard error.

FIG. 8 shows the areas under the curves (AUC) of (A) plasma glucose and (B) serum insulin, after intake of the test sample and the control.

FIG. 9 shows (A) the fasting plasma glucose level and HbA1C level, and (B) the body weight and body fat percentage, at 0, 45, and 90 days after starting a long-term administration test of the test sample.

### BEST MODE FOR CARRYING OUT THE INVENTION

The present invention relates to nutritional compositions for enhancing the production of PPARs and/or PPAR-associated factors, wherein the compositions comprise proteins, fats, and carbohydrates such that proteins account for 10% to 25% of its energy, fats account for 10% to 35% of its energy, and carbohydrates account for 40% to 60% of its energy, and wherein oleate esters account for 60% to 90% of the energy in the fats, and palatinose and/or trehalose account for 60% to 100% of the energy in the carbohydrates. The present invention is based on the present inventors' discovery that nutritional compositions comprising particular compositions have the activity of enhancing the production of PPARs and/or PPAR-associated factors.

In the present invention, PPARs refer to peroxisome proliferator-activated receptors. PPARs are transcription factors belonging to the nuclear receptor superfamily, and mammals have α, γ, and δ subtypes. PPARs form heterodimers with retinoid X receptors (RXR), and induce expression of target genes that have a PPAR-responsive element (PPRE) in their promoter region in a ligand-dependent manner. In the present invention, PPAR-associated factors are factors whose genes comprise a PPRE and which are PPAR-targeted genes whose expression is regulated by any one of the PPAR subtypes, wherein enhancing expression of the factors has the effect of improving insulin resistance or obesity and lipid metabolism. In the present invention, examples of a PPAR-associated factor include fatty acid transport proteins (FATPs), acyl-CoA synthetase (ACS), acyl-CoA oxidase (ACO), peroxisomal bifunctional enzyme (BIFEZ), and carnitine palmitoyltransferase-1 (CPT-1), the genes for which are PPARα target genes, and adiponectin, the gene for which is a PPARγ target gene. The nutritional compositions of the present invention (hereinafter also referred to as “nutritional compositions” or “compositions”) have the activity of enhancing the production of any one or more of the PPAR subtypes and/or any one or more of the PPAR-associated factors, and preferably have the activity of enhancing the production of one or more of PPARα, PPARγ, and adiponectin, and most preferably enhance the production of adiponectin.

The nutritional compositions of the present invention, which have the above-mentioned activity, contain a specific composition of proteins, fats, and carbohydrates. This composition is described in detail below.

Proteins account for 10% to 25%, and preferably 15% to 25%, of the energy in the compositions of the present invention.

Proteins used to prepare the compositions of the present invention include milk proteins, vegetable-derived proteins, soybean proteins, and hydrolysates thereof, but milk proteins are generally used. Examples of milk proteins include MPC (Milk Protein Concentrate), casein protein, whey proteins, magnesium caseinate, hydrolysates thereof, fermented milk, and components obtained by removing whey from fermented milk (such as fresh cheese and quark) (Japanese Patent Application Kokai Publication No. (JP-A) H05-252896 (unexamined, published Japanese patent application)). Of these, MPC is preferably comprised, and compositions comprising both MPC and casein are most preferable.

Examples of whey proteins include whey powders prepared by concentrating and drying whey, whey protein concentrates (WPC) prepared by concentrating whey by ultrafiltration (UF) and then drying it, defatted WPCs prepared by removing fat from whey and then subjecting this to UF concentration (low-fat and high-protein), whey protein isolates (WPI) prepared by selectively separating out only proteins from whey, desalted whey prepared by concentration using nanofiltration, and mineral-concentrated whey prepared by concentrating whey-derived mineral components.

Fats account for 10% to 35%, and preferably 20% to 35%, of the energy in the nutritional compositions of the present invention. This percentage is based on the Sixth Revision of the Nutritional Requirements of the Japanese People. To increase the monounsaturated fatty acid (MUFA) content in the fatty acid composition of the fats, the compositions preferably contain large amounts of oleate esters, which are monounsaturated fatty acid esters. Oleate esters generally account for 60% to 90%, and preferably 60% to 80%, of the energy contained in the fats of the nutritional compositions of the present invention. Fat sources rich in oleic acids include, for example, high oleic sunflower oils, rapeseed oils olive oils, high oleic acid safflower oils, soybean oils, corn oils, and palm oils. Examples of fat sources containing oleate esters include prepared nutritional oils and fats (for example, from the Nippon Oil & Fat Corporation). Sunflower oils, rapeseed oils, olive oils, or a mixture of olive oils and the above-mentioned oils and fats may also be used.

Milk-derived phospholipids and lecithin (derived from soybean or egg yolk) are preferably used as the other lipids.

Milk phospholipids are localized only at the milk fat globule membrane (MFGM) in milk. Examples of substances that are rich in the MGFM include freeze-dried byproducts of WPIs, which are produced by combining ultrafiltration (UF) and microfiltration (MF) (solutions retained by the MF (microfiltration) membrane), and fractions obtained by removing butter oil from whey cream (butter serums). Fat fractions extracted from butter serums several times with ethanol and then concentrated may also be used.

Lecithin chemically means phosphatidylcholine (PC), but ordinarily refers to a mixture of four compounds: PC as well as phosphatidylethanolamine (PE), phosphatidylinositol (PI), and phosphatidic acid (PA), and other phospholipids. In the present invention, all such lecithins may be used. Additional examples are lecithin in the form of pastes containing 62% to 65% of an acetone-insoluble fraction, which is an indicator of phospholipid purity; powdered high-purity lecithin whose phospholipid content is 95% or more; and fractionated lecithin whose phosphatidylcholine content has been increased.

The compositions of the present invention may contain n-6 series polyunsaturated fatty acid esters and n-3 series polyunsaturated fatty acid esters. Preferably, these polyunsaturated fatty acid esters account for 10% to 40%, or more preferably 10% to 30% of the fats. For example, these polyunsaturated fatty acid esters may account for approximately 20% of the fats.

The nutritional compositions of the present invention can blend n-6 series polyunsaturated fatty acid esters with n-3 series polyunsaturated fatty acid esters in ratios of approximately 5:1 to approximately 1:1, or preferably 4:1. To accomplish this, perilla oils (Perilla frutescens oil), linseed oils, and such, which have a high content of n-3 family α-linolenic acid esters, are preferably mixed. Skipjack oils and tuna oils, which are rich in docosahexaenoic acid (DHA), may also be used.

In the present invention, the fats are preferably at least one selected from milk phospholipids, soybean lecithins, high oleic sunflower oils, and perilla oils.

Carbohydrates account for 40% to 60%, and preferably 40% to 55%, of the energy of the nutritional compositions of the present invention. This energy ratio is approximately in line with the Sixth Revision of the Nutritional Requirements of the Japanese People. Palatinose, trehalose, or mixtures thereof are used as carbohydrates. Palatinose, trehalose, or mixtures thereof account for 60% to 100%, and preferably 60% to 80%, of the energy of the carbohydrates.

Examples of other carbohydrates include sugar alcohols (such as sorbitol, xylitol, and maltitol), trehalose, palatinit, maltodextrin, modified starches, amylose starches, tapioca starches, corn starches, fructose, lactose, or mixtures thereof. Of these, maltodextrin, xylitol, or mixtures thereof are preferred. Maltodextrin is a sugar that is an intermediate product obtained by acid hydrolysis or enzymolysis of starch or corn starch, and it has a DE value of 20 or less. The DE (dextrose equivalent) value is an indicator of the hydrolysis rate of starches and sugars, and can be determined from the following equation:

DE=direct reducing sugar (as glucose equivalent)÷solid material×100

The nutritional compositions of the present invention may further include dietary fibers. The dietary fibers may be either water-soluble dietary fibers or water-insoluble dietary fibers. Examples of water-soluble dietary fibers include indigestible dextrin, pectin, glucomannan, alginate and alginate degradation products, guar gum, guar gum enzymolysis product, and galactomannan. Indigestible dextrin is preferred because it can be easily added to foods and does not interfere with food processing. Examples of water-insoluble dietary fibers include crystalline cellulose, soybean dietary fiber, wheat bran, corn fiber, and beet fiber.

The nutritional compositions of the present invention may include vitamins and minerals in accordance with the standard amounts blended in liquid formula. Vitamins include, for example, vitamin B₂, nicotinamide, vitamin B₆, calcium pantothenate, folic acid, vitamin B₁₂, vitamin A fatty acid ester, vitamin D₃, α-vitamin E, vitamin K₂, sodium L-ascorbate, and β-carotene. Minerals include calcium, phosphorus, iron, sodium, potassium, chlorine, magnesium, or trace elements derived from natural products, for example, minerals in yeast such as copper, zinc, selenium, manganese, and chromium. Copper gluconate, zinc gluconate, and such may also be used.

The nutritional compositions of the present invention preferably have an osmotic pressure of approximately 200 to 1000 mOsm/L, and approximately 300 to 750 mOsm/L, for example. The viscosity of the nutritional compositions when measured at 20° C. is preferably about 5 to 40 mPa·s, and in particular, 5 to 20 mPa·s.

The nutritional compositions preferably provide about 0.5 to 3 kcal/mL, and in particular, 1 to 1.5 kcal/mL.

The nutritional compositions are preferably in forms enabling direct intake. In these forms, the compositions can be taken orally, or from the nose to the stomach and jejunum through a tube. The nutritional compositions of the present invention may take a variety of forms, for example, they may be juice-type drinks or milkshake-type drinks, so long as they maintain the above-mentioned composition. The nutritional compositions can also be made into soluble powders that can be reconstituted before use.

The nutritional compositions may include various flavors (for example, vanilla and such), sweeteners, and other additives. Artificial sweeteners, for example aspartame and such, may be used.

Champignon extract, which has a deodorizing effect on fecal odor, can be included at 5 mg to 500 mg (0.005% to 0.5%); and carotinoid formulations (including, for example, α-carotene, β-carotene, lycopene, and lutein) can also be included at 10 μg to 200 μg (0.00001% to 0.0002%) for the purpose of nutritional enhancement.

Catechin, polyphenols, and such may also be included as antioxidants.

The nutritional compositions can be produced, for example, by mixing proteins, fats, and carbohydrates in the ratios described above. Herein, emulsifiers can be blended into the mixture.

The nutritional compositions of the present invention can be prepared as products by methods well known in the art. Such methods include, for example, methods of heat-sterilizing liquid nutritional compositions, then aseptically filling them into containers (for example, methods using both UHT sterilization methods and aseptic packaging); or methods of filling liquid nutritional compositions into containers, and then heat-sterilizing the compositions along with the containers (for example, autoclave methods).

When the compositions are used in a liquid form, homogenates are filled into can containers and then retort-sterilized, or are heat-sterilized again at approximately 140-145° C. for approximately 5-8 seconds, cooled, and then aseptically filled into containers. When the compositions are used in a powder form, homogenates may be spray dried, for example. When the compositions are used in a solid form, agar or such may be added for solidification.

The ability of the nutritional compositions produced as described above to enhance the production of PPARs and/or PPAR-associated factors can be confirmed using known methods. As described below in the Examples, in one example the mRNAs of a PPAR and/or PPAR-associated factor can be quantified by extracting total RNAs from the liver or visceral fat of animals that have ingested a test substance, and then using RT-PCR using primers specific to the genes of the PPAR and/or PPAR-associated factor. Examples of the sequences of the above-mentioned primers are shown in Table 7 and in SEQ ID NOs: 1 to 32. Highly quantitative results can be expected by employing real-time PCR. If the expression levels of PPAR and/or PPAR-associated factors in the test substance group are increased compared to the control group, then this test substance is judged to have the activity of enhancing the production of PPAR and/or PPAR-associated factors. Alternatively, such an evaluation can be made by using an antibody method to measure the PPAR and/or PPAR-associated factors in samples such as urine, protein fractions or blood (plasma, serum, etc.) extracted from the liver or adipose tissues of animals that have ingested a test substance, using antibodies specific to PPAR and/or PPAR-associated factors, and then comparing the measurements with a control. Antibody methods can be selected from known immunoassay methods such as RIA, EIA, ELISA, CLEIA, and CLIA. The samples can be subjected to electrophoresis and then to Southern hybridization, and the bands can be quantified and then compared to a control for evaluation.

As described above, the nutritional compositions of the present invention enhance the production of PPARs and/or PPAR-associated factors. Due to the ameliorating effects of PPARs and PPAR-associated factors on insulin resistance and obesity, the nutritional compositions of the present invention are useful as foods for treating or preventing impaired glucose tolerance, type II diabetes, and obesity. As described below in the Examples, the results of administering the nutritional compositions of the present invention to healthy individuals showed that the compositions have the activity of lowering blood glucose levels, serum insulin concentrations, and free fatty acid concentrations in serum. Further, long term administration to human patients with impaired glucose tolerance (IGT) resulted in actual reductions in fasting blood glucose levels, HbA1c levels, body weights, and body, fat percentages. Thus, the utility of the compositions as therapeutic or preventive foods for type II diabetes and such has been proven. In addition, the compositions are expected to be effective for treating or preventing metabolic diseases that develop based on insulin resistance, such as hyperlipidemia, hypertension, and arteriosclerosis.

In the area of neurosurgery, many patients have impaired consciousness and are incapable of voluntary feeding behaviors; further, patients 40 years old or older, who are of middle and advanced age, often have some kind of concurrent illness. In many cases the ability of such patients with impaired consciousness to digest and absorb food is not inhibited, and nutrients can be administered via the intestinal tract, which is a more physiological route for dietary intake. Therefore, the nutritional compositions of the present invention play an important role in terms of nutritional management. Further, patients with multiple organ dysfunction syndrome (MODS) as well as renal failure can easily develop water and electrolyte disorders, which interfere with enteral alimentation from the early stages. Such cases require liquid nutritional compositions that take account of water and electrolytes in kidney failure. The nutritional compositions of the present invention also hold much promise as such nutritional compositions.

In addition to their use as oral and tube nutritional agents, therapeutic foods, and foods for patients at home, the nutritional compositions of the present invention may also be used as foods that show an effect in improving serum lipid metabolism, or an effect in decreasing blood sugar levels, such as Foods with Health Claims (Foods for Specified Health Uses, and Foods with Nutrient Function Claims).

Administration of a nutritional composition to a patient differs depending on the patient's condition, body weight, and age, whether the nutritional composition is the only source of nutrition, and so on. The dose is determined by the physician in charge of the patient. When a nutritional composition is used as a supplement in other foods, the amount of the nutritional composition administered in a day is reduced accordingly.

A nutritional composition of the present invention can be taken in multiple administrations, for example two to five times, to supplement the daily requirement, or can be taken in one administration. A nutritional composition may be supplied continuously over the period required.

Agar can be added to the liquid nutritional compositions, or water and agar can be added to powdered nutritional compositions, and after heat treatment followed by cooling, the nutritional compositions can be taken as solid nutritional compositions. Since solid nutritional compositions can give a feeling of fullness after intake, they can be taken as a substitute for ordinary solid foods.

All prior art references cited herein are incorporated herein by reference.

### EXAMPLES

Hereinbelow, the present invention will be specifically described with reference to Examples and Test Examples, but it is not to be construed as being limited thereto.

### Example 1

A liquid nutritional composition was prepared as per the quantities of ingredients shown in Table 1, below. This composition had 100 kcal/100 mL, and its energy ratio was 23.7% protein, 30.2% fat, and 46.1% carbohydrate. Oleate esters accounted for 70% of the energy in the fats, and palatinose accounted for 69% of the energy in the carbohydrates. This resulted in effects equivalent to the nutritional compositions in the Test Examples below.

The milk protein concentrate that was used came from Fonterra, New Zealand; the caseinate came from DMV; the milk phospholipids from New Zealand Dairy Ingredients Limited; the indigestible dextrin from Matsutani Chemical Industry; the high oleic sunflower oil from Nippon Oil & Fat Corporation (oleic acid content 80%); the Perilla frutescens oil from Nippon Oil & Fat Corporation (6% palmitic acid, 2% stearic acid, 19% oleic acid, 12% linoleic acid, and 60% α-linolenic acid); and the palatinose from Mitsui Sugar Co.

TABLE 1

| | | Basic Formulation | |
|---|---|---|---|
| Components | Ingredients | per 100 g | |
| Protein | Milk protein concentrate (MPC) | 5 | g |
| | Caseinate | 1 | g |
| Fat | Nutritional prepared oils and fats | 3.0 | g |
| | (containing 10% perilla oil) | | |
| | Milk phospholipids | 0.1 | g |
| | Soybean lecithin | 0.3 | g |
| Carbohydrate | Palatinose | 8 | g |
| | Maltodextrin | 3 | g |
| | Xylitol | 0.9 | g |
| Dietary fiber | Indigestible dextrin | 1.6 | g |
| General | Flavors | 0.5 | g |
| components | Citric acid (pH adjuster) | 0.2 | g |
| Vitamin | Vitamin A fatty acid ester | 1.3 | mg |
| | Vitamin D₃ | 0.005 | mg |
| | α-Vitamin E (α-TE) | 40 | mg |
| | Dibenzoyl thiamine hydrochloride | 4.7 | mg |
| | Vitamin B₂ | 2.6 | mg |
| | Vitamin B₆ | 3.7 | mg |
| | Vitamin B₁₂ | 0.005 | mg |
| | Niacin | 29.4 | mg |
| | Pantothenic acid | 9.5 | mg |
| | Folic acid | 0.49 | mg |
| | Vitamin C | 60.6 | mg |
| | Vitamin K₂ | 0.11 | mg |
| | α-Carotene | 0.8 | μg |
| | β-Carotene | 4.2 | μg |
| | Lutein | 1.4 | μg |
| | Lycopene | 5.59 | μg |
| Mineral | Sodium chloride | 100 | mg |
| | Potassium hydroxide | 150 | mg |
| | Magnesium sulfate heptahydrate | 10 | mg |
| | Trisodium citrate dihydrate | 120 | mg |
| | Ferrous sulfate | 5 | mg |

### Example 2

A liquid nutritional composition was prepared as per the quantities of ingredients shown in Table 2, below. This nutritional composition had 100 kcal/100 mL, and its energy ratio was 24% protein, 30% fat, and 46% carbohydrate. Oleate esters accounted for 70% of the energy in the fats, and palatinose accounted for 69% of the energy in the carbohydrates. This resulted in effects equivalent to the nutritional compositions in the Test Examples below.

TABLE 2

| | | Basic Formulation | |
|---|---|---|---|
| Components | Ingredients | per 100 g | |
| Protein | Milk protein concentrate (MPC) | 3.5 | g |
| | Caseinate | 2.4 | g |
| Fat | High oleic sunflower oil + perilla | 2.91 | g |
| | oil | | |
| | Milk phospholipids | 0.1 | g |
| | Soybean lecithin | 0.29 | g |
| Carbohydrate | Palatinose | 7.01 | g |
| | Maltodextrin | 2.45 | g |
| | Xylitol | 0.9 | g |
| Dietary fiber | Indigestible dextrin | 1.88 | g |
| General | Flavors | 0.38 | g |
| components | Champignon extract | 0.05 | g |
| | Citric acid (pH regulator) | 0.13 | g |
| Vitamin | Vitamin A | 250 | IU |
| | Vitamin D | 30 | IU |
| | Vitamin E (α-TE) | 13.1 | mg |
| | Vitamin B₁ | 0.96 | mg |
| | Vitamin B₂ | 0.6 | mg |
| | Vitamin B₆ | 0.4 | mg |
| | Vitamin B₁₂ | 1.1 | μg |
| | Niacin | 1.8 | mg |
| | Pantothenic acid | 1.2 | mg |
| | Folic acid | 75 | μg |
| | Vitamin C | 91 | mg |
| | α-Carotene | 0.8 | μg |
| | β-Carotene | 4.2 | μg |
| | Lutein | 1.4 | μg |
| | Lycopene | 5.6 | μg |
| Mineral | Sodium chloride | 100 | mg |
| | Ferrous sulfate | 5 | mg |
| | Chromium yeast | 2 | mg |
| | Zinc yeast | 5 | mg |
| | Potassium dihydrogen phosphate | 20 | mg |
| | Trisodium citrate dihydrate | 100 | mg |
| | Potassium hydroxide | 100 | mg |

### Example 3

A liquid nutritional composition was prepared as per the quantities of ingredients shown in Table 3, below. This nutritional composition had 100 kcal/100 mL, and its energy ratio was 22% protein, 30% fat, and 48% carbohydrate. Oleate esters accounted for 70% of the energy in the fats, and palatinose accounted for 69% of the energy in the carbohydrates. This resulted in effects equivalent to the nutritional compositions in the Test Examples below.

TABLE 3

| | | Basic Formulation | |
|---|---|---|---|
| Components | Ingredients | per 100 g | |
| Protein | Milk protein concentrate (MPC) | 3.2 | g |
| | Caseinate | 2.4 | g |
| Fat | High oleic sunflower oil + perilla | 2.9 | g |
| | oil | | |
| | Milk phospholipids | 0.1 | g |
| | Soybean lecithin | 0.29 | g |
| Carbohydrate | Palatinose | 8 | g |
| | Maltodextrin | 3 | g |
| | Xylitol | 0.9 | g |
| Dietary fiber | Indigestible dextrin | 1.5 | g |
| General | Flavors | 0.4 | g |
| components | Champignon extract | 0.05 | g |
| Vitamin | Vitamin A | 250 | IU |
| | Vitamin D | 30 | IU |
| | Natural vitamin E (α-TE) | 8 | mg |
| | Vitamin B₁ | 0.6 | mg |
| | Vitamin B₂ | 0.5 | mg |
| | Vitamin B₆ | 0.3 | mg |
| | Vitamin B₁₂ | 0.9 | μg |
| | Niacin | 1.6 | mg |
| | Calcium pantothenate | 1.0 | mg |
| | Folic acid | 50 | μg |
| | Vitamin C | 45 | mg |
| | α-Carotene | 0.8 | μg |
| | β-Carotene | 4.2 | μg |
| | Lutein | 1.4 | μg |
| | Lycopene | 5.6 | μg |
| Mineral | Sodium chloride | 100 | mg |
| | Potassium hydroxide | 100 | mg |
| | Potassium dihydrogen phosphate | 20 | mg |
| | Chromium yeast | 2 | mg |
| | Zinc yeast | 5 | mg |
| | Trisodium citrate dihydrate | 100 | mg |
| | Ferrous sulfate | 5 | mg |

### Example 4

### Preparation of a Powdered Nutritional Composition

53 kg of a liquid nutritional composition was prepared as per the quantities of ingredients shown in Table 3, and this was concentrated to 32 kg using an evaporator. This concentrated nutritional composition was treated in a spray dryer (exhaust air temperature: 95° C.; orifice No. 74; and core No. 17) to obtain 10 kg of powdered nutritional composition. Meibalance C (hereinafter also referred to as MBC) (Table 4), and Glucerna (Table 5) which were used as controls were similarly treated to obtain powders. The solid contents of the powdered nutritional composition, Glucerna, and Meibalance C were 96.7%, 95.3%, and 96.3%, respectively. The energy per gram of material was 5.6 kcal for the powdered nutritional composition, 5.5 kcal for Glucerna, and 4.6 kcal for powdered Meibalance C.

TABLE 4

| | | Basic Formulation | |
|---|---|---|---|
| Components | Ingredients | per 100 g | |
| Protein | Milk protein concentrate (MPC) | 4 | g |
| Carbohydrate | Dextrin | 14.2 | g |
| | Sucrose | 0.4 | g |
| Fat | Vegetable oil | 2.8 | g |
| Dietary Fiber | Indigestible dextrin | 1 | g |
| Mineral | Potassium | 100 | mg |
| | Sodium | 110 | mg |
| | Chlorine | 140 | mg |
| | Calcium | 110 | mg |
| | Phosphorus | 85 | mg |
| | Magnesium | 15 | mg |
| | Iron | 1 | mg |
| Vitamin | Vitamin A | 200 | IU |
| | Vitamin D | 20 | IU |
| | Vitamin E | 3 | mg |
| | Vitamin B₁ | 0.15 | mg |
| | Vitamin B₂ | 0.2 | mg |
| | Vitamin B₆ | 0.3 | mg |
| | Vitamin B₁₂ | 0.6 | μg |
| | Niacin | 1.6 | mg |
| | Pantothenic acid | 0.6 | mg |
| | Folic acid | 50 | μg |
| | Vitamin C | 16 | mg |

TABLE 5

| | | Basic Formulation | |
|---|---|---|---|
| Components | Ingredients | per 100 g | |
| Protein | Casein | 4.2 | g |
| Carbohydrate | Maltodextrin | 6.2 | g |
| | Fructose | 1.7 | g |
| Fat | Sunflower oil + Soybean oil + | 5.56 | g |
| | Soybean lecithin | | |
| Dietary Fiber | Soybean polysaccharides | 1.4 | g |
| Mineral | Potassium | 156 | mg |
| | Sodium | 93.2 | mg |
| | Chlorine | 144 | mg |
| | Calcium | 70 | mg |
| | Phosphorus | 70 | mg |
| | Magnesium | 28 | mg |
| | Iron | 1.4 | mg |
| Vitamin | Vitamin A | 352 | IU |
| | Vitamin D | 28 | IU |
| | Vitamin E | 3.2 | IU |
| | Vitamin B₁ | 0.16 | mg |
| | Vitamin B₂ | 0.18 | mg |
| | Vitamin B₆ | 0.22 | mg |
| | Vitamin B₁₂ | 0.64 | mg |
| | Niacin | 2.12 | mg |
| | Pantothenic acid | 0.92 | mg |
| | Folic acid | 42 | mg |
| | Vitamin C | 21.2 | mg |

### Example 5

### Procedure for Solidifying the Nutritional Composition

Two grams of agar (product name: “Kanten Kukku”, Ina Food Industry) were added to 120 g of the powdered nutritional composition prepared in Example 4. 150 mL of hot (approximately 60° C.) water was added and then mixed. This mixture was heated in a 500 watt high frequency microwave oven (RE-BM5W, SAMSUNG) for five minutes, and then solidified in a refrigerator. This nutritional composition has 672 kcal. The number of calories in the nutritional composition can be adjusted to the required number of calories. The agar concentration is preferably 0.5% to 2%.

### Test Example 1

### 1. Production of Laboratory Animals and Feed

19-week old male Sprague-Dawley rats (Japan SLC) were purchased and bred in the animal laboratory (specific-pathogen free, room temperature of 23±1° C., 12-hour light-dark cycle) of the Institute for Animal Experimentation at the University of Tokushima, following the Guidelines for Care and Use of Lab Animals of the University of Tokushima. For one week after purchase, they were freely allowed to drink water and feed on standard solid feed for breeding rats (MF-type, Oriental Yeast Co.).

After fasting for 24 hours on the day before starting the experiment, 2.0 mL of blood was collected for biochemical tests from the left jugular vein under diethyl ether anesthesia. The rats were then randomly divided into three groups (n=3): the MF group, MBC group, and test sample group. The composition of the test sample is shown in Table 6. The test sample had a caloric ratio (protein/fat/carbohydrate) of 20%/29.7%/50.3% and contained 0.1 g/100 mL of milk phospholipid extract, 2.4 g/100 mL of oleic acid, and 7.0 g/100 mL of palatinose as carbohydrate.

TABLE 6

| | | | per 100 mL |
|---|---|---|---|
| Energy | | kcal | 100 |
| Protein | | g | 5.0 |
| Fat | | g | 3.3 |
| Carbohydrate | | g | 12.4 |
| Dietary fiber | | g | 1.5 |
| Ash | | g | 0.7 |
| Water | | g | 84.2 |
| Vitamin | Vitamin A | μg RE*¹ | 75 (250) |
| | | (IU) | |
| | Vitamin D | μg | 0.75 (30) |
| | | (IU) | |
| | Vitamin E | mg α-TE*² | 8.0 |
| | Vitamin K | μg | 1* |
| | Vitamin B₁ | mg | 0.60 |
| | Vitamin B₂ | mg | 0.50 |
| | Niacin | mg NE*³ | 2.1 |
| | | mg | 1.6 |
| | Vitamin B₆ | mg | 0.30 |
| | Folic acid | μg | 50 |
| | Vitamin B₁₂ | μg | 0.9 |
| | Biotin | μg | 0.29* |
| | Pantothenic acid | mg | 1.00 |
| | Vitamin C | mg | 40 |
| | Choline | mg | 19.4* |
| Mineral | Sodium | mg | 70 (30.4 mEq/L) |
| | (equivalent of | g | 0.18 |
| | sodium chloride) | | |
| | Calcium | mg | 80 (20.0 mmol/L) |
| | Iron | mg | 1.0 |
| | Phosphorus | mg | 80 (25.8 mmol/L) |
| | Magnesium | mg | 25 (10.3 mmol/L) |
| | Potassium | mg | 80 (20.5 mEq/L) |
| | Copper | mg | 0.02* |
| | Iodine | μg | 2.8* |
| | Manganese | mg | 0.01* |
| | Selenium | μg | 2.8* |
| | Zinc | mg | 0.8 |
| | Chromium | μg | 3 |
| | Molybdenum | μg | 2.9* |
| | Chlorine | mg | 60 (16.9 mEq/L) |

*¹Retinol equivalent
*²α-Tocopherol equivalent
*³Niacin equivalent

The experiment was eight weeks long, and the MF diet, MBC diet, or test sample diet was given to each group such that food intake was 70 to 80 kcal/day. The MBC and test sample, which are liquid diets, were spray dried and the resulting powders were given to the animals.

### 2. Dissection

Immediately after this experimental period was completed, the animals were fasted for 24 hours and then dissected under nembutal anesthesia (0.8″mL/kg B.W.). The liver and visceral adipose tissues (mesenteric fat, epididymal fat, and retroperitoneal fat) were removed for RNA extraction. Epididymal fat was the visceral adipose tissue used for RNA extraction.

### 3. RNA Extraction and cDNA Synthesis

Total RNAs were extracted from the removed liver and epididymal fat sections using a tenfold amount of ISOGEN (Nippon Gene) according to the manufacturer's protocols. To synthesize the cDNAs, the equivalent of 5 μg of the extracted RNAs was reacted with 150 μg of Random Primers (Invitrogen), 2.5 mM dNTP mixture (Takara Bio Inc.), 5× First Strand Buffer (Invitrogen), 0.1 M DTT (Invitrogen), and 400 U of M-MLV Reverse Transcriptase (Invitrogen) in a system with a total volume of 50 μL.

### 4. Real-Time Quantitative PCR

The expression level of each gene was quantified by real-time PCR on a Light Cycler™ (Roche Diagnostics) using 2× QuantiTect™ SYBR Green PCR Master Mix (QIAGEN). The Mg concentration in the reaction solution was 3.0 mM. The PCR reaction conditions were 50 cycles of “incubation at 95° C. for 15 minutes, denaturation at 95° C. for ten seconds, annealing at 60° C. for 15 seconds, and extension at 72° C. for 15 seconds”. Amplification reactions were performed using 1 μL of the cDNAs prepared in 3. as templates, and primers specific for each gene (SEQ ID NOs: 1 to 32, and Table 7). Melting Curve Analysis confirmed that a single PCR product was obtained.

TABLE 7

| | GenBank | | Product |
|---|---|---|---|
| Name | accession No. | Sequence (5′ → 3′) | size |
| PPAR α | M88529 | F tgtatgaagccatcttcacg | 163 bp |
| peroxisome proliferator- | | R ggcattgaacttcatagcga | |
| activated receptor α | | | |
| PPAR γ | AF156665 | F tcaaaccctttaccacggtt | 147 bp |
| peroxisome proliferator- | | R caggctctactttgatcgca | |
| activated receptor γ | | | |
| SREBP-1c | AF286470 | F ggagccatggattgcacattt | 190 bp |
| sterol regulatory-element | | R tccttccgaaggtctctcctc | |
| binding protein 1c | | | |
| HSL | X51415 | F agagccatcagacagccccgagat | 229 bp |
| hormone sensitive lipase | | R tgacgagtagaggggcatgtggag | |
| FATP | U89529 | F aggtgacgtgctagtgatgg | 100 bp |
| fatty acid transport | | R ctccgtggtggatacgttct | |
| protein | | | |
| very long-chain ACS | D85100 | F gtgcggttcttcctgcaact | 134 bp |
| very-long-chain acyl-CoA | | R aacagcaggaagggcttgtg | |
| synthetase | | | |
| ACO | J02752 | F atggcagtccggagaataccc | 114 bp |
| acyl-CoA oxidase | | R cctcataacgctggcttcgagt | |
| BIFEZ | K03249 | F aggtcattcctagccgatac | 185 bp |
| peroxisomal bifunctional | | R tacatcctctggcttgctac | |
| enzyme | | | |
| long-chain ACS | D90109 | F atcaggctgcttatggatga | 116 bp |
| long-chain acyl-CoA | | R ttcactgacgtgtttgcttg | |
| synthetase | | | |
| CPT-1 | NM_031559 | F ggtgggccacaaattacgtg | 104 bp |
| carnitine palmitoyl- | | R cagcatctccatggcgtagt | |
| transferase 1 | | | |
| DCI | NM_017306 | F tccgaggtgtcatcctcact | 115 bp |
| 3-2 trans Enoyl-CoA | | R tgcacagccttccagtactc | |
| isomerase | | | |
| FAS | M76767 | F tgggcccagcttcttagcc | 104 bp |
| fatty acid synthase | | R ggaacagcgcagtaccgtaga | |
| UCP2 | NM_019354 | F tctcccaatgttgcccgaaa | 107 bp |
| uncoupling protein 2 | | R gggaggtcgtctgtcatgag | |
| Acrp30 (Adiponectin) | NM_144744 | F ggaaacttgtgcaaggttgga | 140 bp |
| adipocyte complement | | R ggtcacccttaggaccaaga | |
| related protein of 30 kDa | | | |
| TNF α | NM_012675 | F atggatctcaaagacaacca | 143 bp |
| tumor necrosis factor | | R tcctggtatgaaatggcaaa | |
| superfamily, member2 | | | |
| β-actin | NM_031144 | F gtcccagtatgcctctggtcgtac | 171 bp |
| β-actin | | R ccacgctcggtcaggatcttcatg | |

The liver was examined for PPARα, PPARγ, and sterol regulatory-element binding protein-1c (SREBP-1c), which are transcription factors, and hormone sensitive lipase (HSL), fatty acid transport protein (FATP), very long-chain acyl-CoA synthetase (very long-chain ACS), acyl-CoA oxidase (ACO), peroxisomal bifunctional enzyme (BIFEZ), long-chain acyl-CoA synthetase (long-chain ACS), carnitine palmitoyl transferase-1 (CPT-1), 3-2 trans enoyl-CoA isomerase (DCI), and fatty acid synthase (FAS), which are lipid metabolism genes. Uncoupling protein 2 (UCP2) was also analyzed in relation to energy consumption. Adipose tissues were examined for the transcription factors PPARα, PPARγ, and SREBP-1c; the adipocytokines Acrp30 (adipocyte complement related protein of 30 kDa: adiponectin) and TNFα (tumor necrosis factor superfamily, member 2); and UCP2 for energy consumption. The expression level of each gene was corrected with β-actin, and all values are indicated as relative values, where the average value of the MF group is defined as 100%.

### 5. Statistical Treatment

The results were presented as mean±standard error (mean±SE), and significant difference tests were performed between the groups using one factor analysis of variance (one-factor ANOVA). Student's t-tests were also performed, and p<0.05 was defined as significant.

### <Results>

### 1. Liver

### (Transcription Factors)

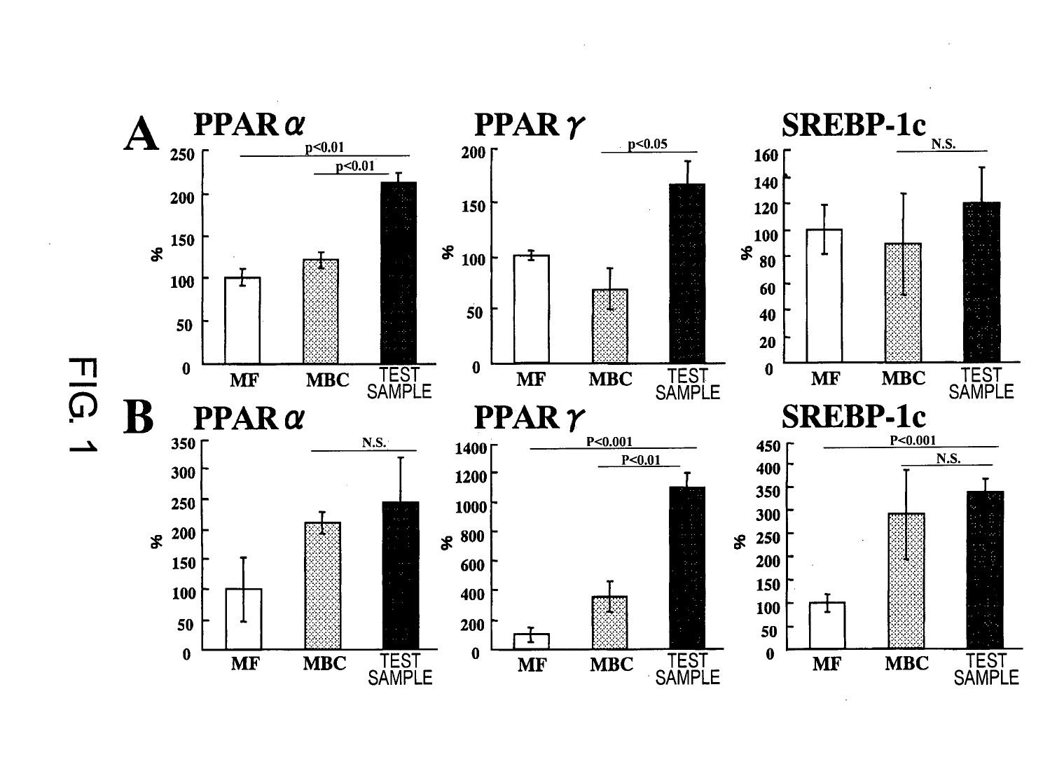

Expression levels of PPARα, PPARγ, and SREBP-1c, thought to be particularly important transcription factors involved in lipid metabolism, were examined. The expression levels of PPARα and PPARγ in the liver of the test sample group were respectively about twice and about 2.5 times that in the MBC group, showing significantly enhanced expression (PPARα: p<0.01, PPARγ: p<0.05) (FIG. 1A). The expression level of SREBP-1c in the test sample group was not significantly different from that in the MBC and MF groups (FIG. 1A).

### (Metabolic Enzymes)

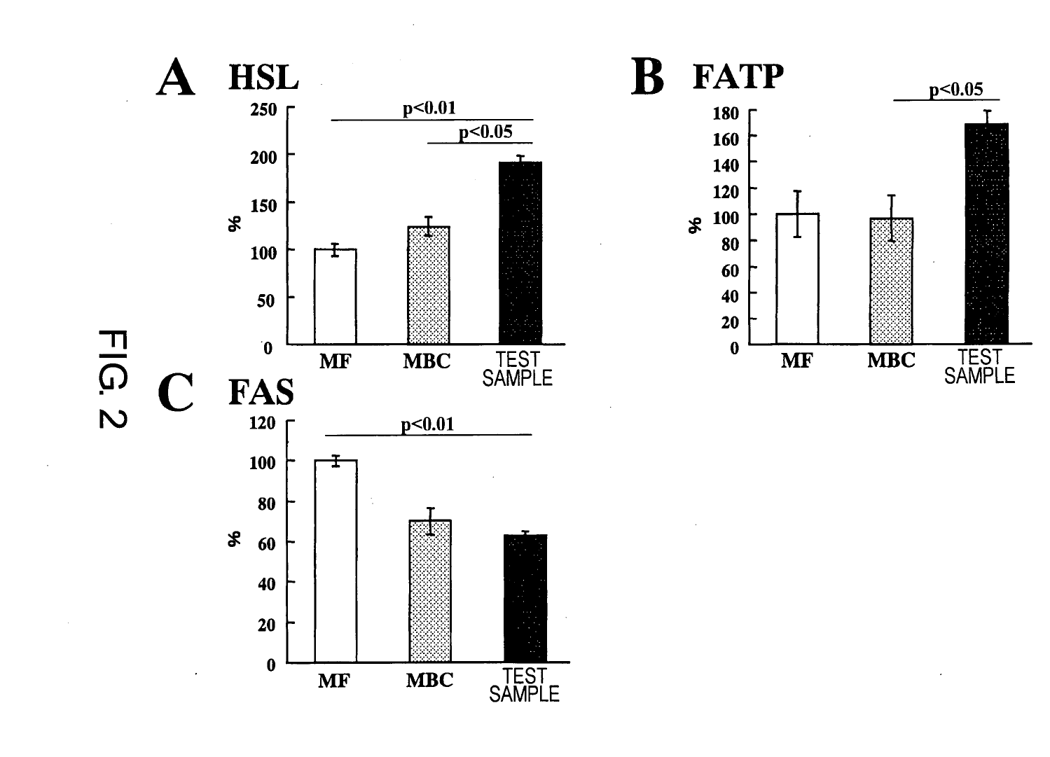

HSL, which is involved in the degradation of stored TG, and FATP, a fatty acid transport protein, both showed significantly higher expression levels in the test sample group than in the MBC group (p<0.05 for both HSL and FATP) (FIGS. 2A and B). In the test sample group, the expression level of FAS, which controls fatty acid synthesis in the liver, was not significantly different from that in the MBC group, but was significantly (P<0.01) lower than the level in the MF group (FIG. 2C).

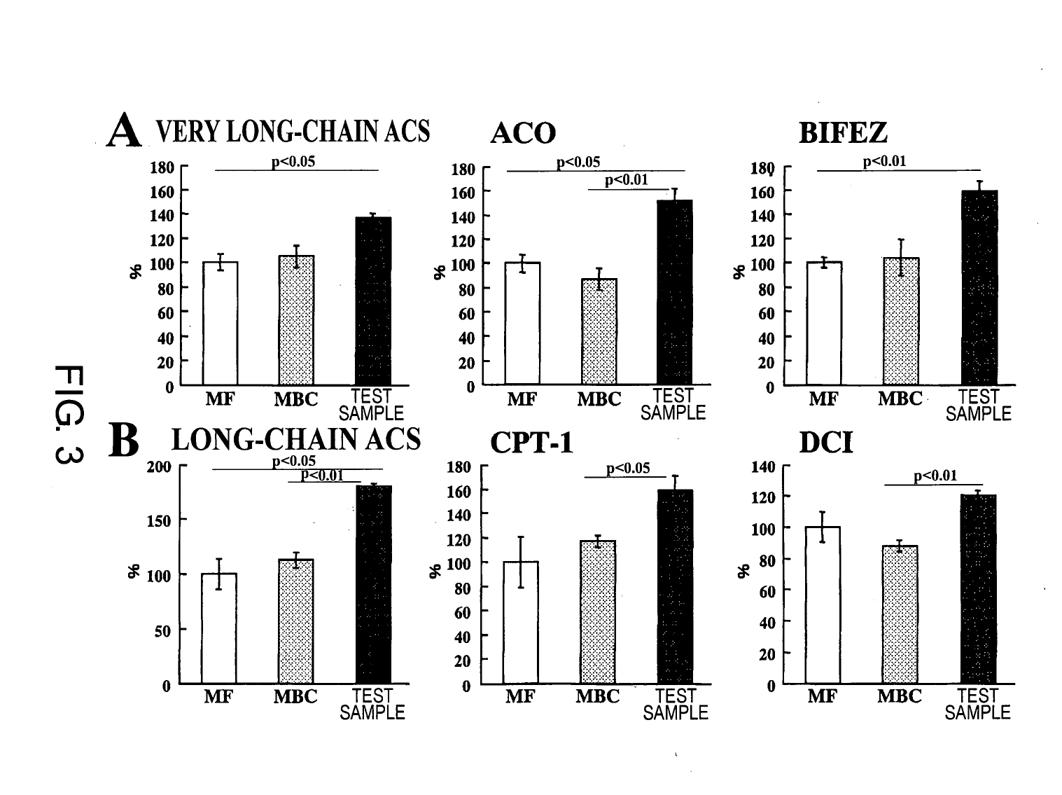

In the peroxisomal β-oxidation system, expression levels of very long-chain ACS, ACO, and BIFEZ were examined. The expression level of ACO in the test sample group was approximately twice that of the MBC group, showing significantly enhanced expression (p<0.01). The expression levels of very long-chain ACS and BIFEZ in the test sample group showed a tendency to be higher than in the MBC group (very long-chain ACS: p=0.069; BIFEZ: p=0.075), and were significantly (very long-chain ACS: p<0.05; BIFEZ: p<0.01) higher than those in the MF group (FIG. 3A). In the mitochondrial β-oxidation system, the expression levels of long-chain ACS, CPT-1, and DCI were analyzed; expression levels were all significantly higher (long-chain ACS and DCI: p<0.01, CPT-1: p<0.05) in the test sample group than in the MBC group (FIG. 3B).

Of the genes associated with lipid metabolism that were examined this time, the target genes carrying PPRE in their promoter region were FATP, ACS, ACO, BIFEZ, and CPT-1, and in each of the groups they showed expression patterns similar to that of PPARα. These PPARα target genes are involved in the fatty acid metabolic pathway, including TG degradation, fatty acid transport, and peroxisomal and mitochondrial β-oxidation, and activation of this pathway is considered to promote the use of fatty acids. This therefore suggests that enhancement of PPARα expression is the key to the test sample's lipid metabolism-improving effect. In addition, PPARγ expression was also significantly increased in the test sample group. Virtually nothing is understood of the physiological activity of PPARγ in the liver, but there are reports that PPARγ induces the expression of glucokinase (GK), which is the rate-limiting enzyme in the glycolysis system. In fact, GK expression was significantly increased in the test sample group (data not shown). This may also suppress the increase of blood glucose.

### (Energy Consumption)

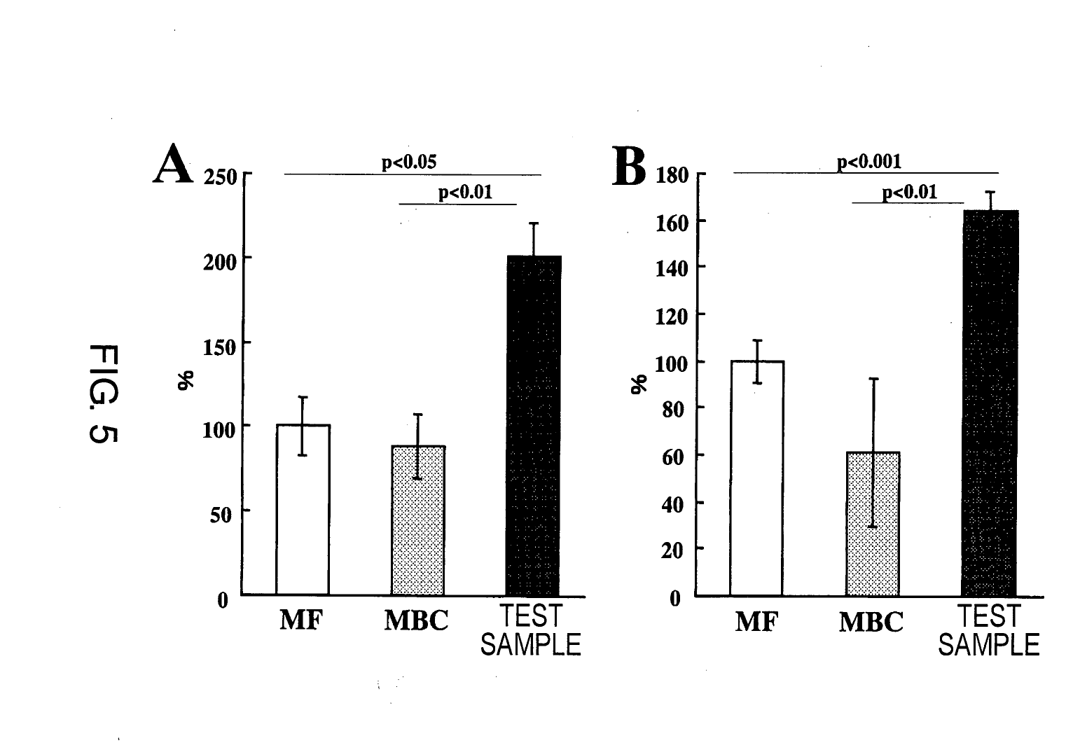

The expression level of UCP2 in the liver of the test sample group was approximately three times that in the MBC group, showing significantly enhanced expression (p<0.01) (FIG. 5A).

### 2. Adipose Tissues

### (Transcription Factors)

In adipose tissues, the expression level of PPARγ in the test sample group was significantly increased to approximately three times that in the MBC group (p<0.01). A significant difference could not be observed for PPARα (FIG. 1B). The level of SREBP-1c expression in the test sample group was not significantly different to that in the MBC group, but was significantly (p<0.001) enhanced compared to that in the MF group (FIG. 1B).

As described above, enhanced expression of PPARα and γ was confirmed in the liver of the test sample group, and enhanced expression of PPARγ was confirmed in the adipose tissues. The activity of PPAR is regulated in a ligand-dependent manner; oleic acid, which is a monounsaturated fatty acid, and α-linolenic acid, which is a n-3 series polyunsaturated fatty acid, are both abundant in the test sample, and have been reported as ligands of both PPARα and γ when at the concentration levels found in human serum. Therefore, the lipid metabolism-improving effect in the test sample group may have been due not only to the increased expression of PPARα and γ, but also to enhancement of their physiological activity by the ligand action of the fatty acids. Due to their chemical properties, fatty acids may function as ligands of various other nuclear receptors in addition to PPARs, and they have been reported to regulate insulin secretion via an orphan receptor, the G-protein-coupled receptor 40 (GPR40), which exists on the membrane of pancreatic β cells. Thus, further investigation is required of the possibility that the characteristic fatty acid composition of the test sample may be exerting some specific effects on the nuclear and membrane receptors that take part in regulation of metabolism.

The possibility that PPARγ expression may be directly induced by the components of the test sample is suggested since: enhanced PPARγ expression was only observed in adipose tissues, even though expression of both PPARα and γ were enhanced in the liver; and since the test sample group and the MBC group showed no difference in the expression level of SREBP-1c, which is, like the PPARs, an important transcription factor for regulating lipid metabolism, and which targets ACS and FAS genes, and whose expression is regulated by unsaturated fatty acids. Thiazolidine agents, which are PPARγ agonists, are known to exhibit the pharmacological action of improving insulin resistance through high-level activation of PPARγ. In the test sample group, as a result of high-level PPARγ activation, differentiation of preadipocytes into small adipocytes may have been induced, and this may have lead to increased adiponectin expression and secretion, which may have caused enhanced expression of PPARα, in the liver as a secondary effect.

### (Adipocytokines)

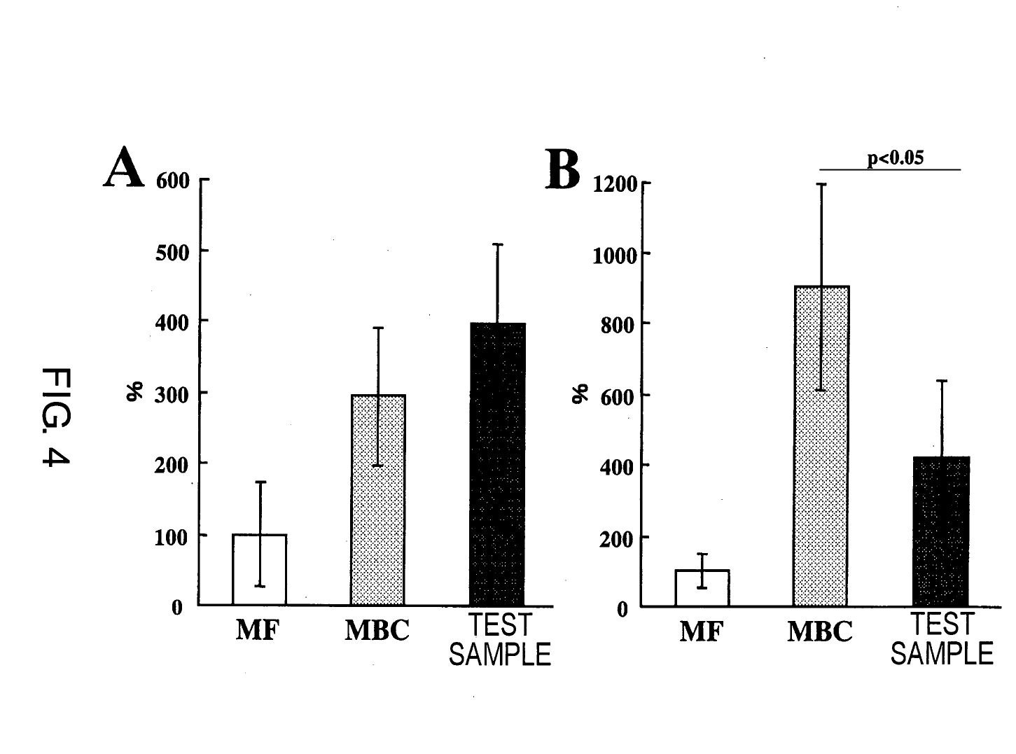

Adiponectin has the activity of improving insulin resistance, and its expression level in the test sample group tended to be higher than in the MBC group, and was significantly (p<0.05) higher than in the MF group (FIG. 4A). On the other hand, the expression level of TNFα, which worsens insulin resistance, was one half or less in the test sample group as compared to in the MBC group, showing significantly (p<0.05) suppressed expression (FIG. 4B).

As mentioned above, in adipose tissues, the expression of adiponectin, which is an adipocytokine that improves insulin resistance, was enhanced and the expression of TNFα, which has the opposing effect, was shown to be decreased. This suggests that systemic insulin sensitivity was increased. It is known that adiponectin is secreted from small adipocytes while TNFα is secreted from adipocytes that have enlarged due to obesity. Adipocytes become smaller not only due to reduced body fat due to weight loss, but also due to promotion of differentiation from preadipocytes via high-level PPARγ activation. Observation of reduced visceral fat and enhanced PPARγ expression in the test sample group strongly suggests a reduction in adipocyte size. Recently, PPRE was found to exist in the promoter region of adiponectin, and binding of PPARγ was found to induce its expression. Further, there are reports that adiponectin acts on the liver to induce PPARα expression, and activates its endogenous ligand action. Enhanced expression of PPARα in the liver in the test sample group is also considered to be an effect of adiponectin arising from PPARγ activation, suggesting that such adiponectin-mediated interaction between the liver and adipose tissues plays an important role in regulating systemic lipid metabolism.

### (Energy Consumption)

The UCP2 expression level in adipose tissues in the test sample group was approximately 2.5 times that of the MBC group, showing significantly enhanced expression (p<0.01) (FIG. 5B).

As described above, UCP2 expression was significantly increased in both the liver and adipose tissues. UCP2 has the function of dissipating energy as heat by uncoupling oxidative phosphorylation in the mitochondrial inner membrane, and acts to promote energy consumption. This may have enhanced systemic energy consumption and effectively suppressed lipid accumulation in rats receiving long-term administration of the test sample. In pancreatic 3 cells UCP2 has also been demonstrated to suppress insulin secretion caused by a high-fat diet or glucose stimulus. This may be contributing to the reduced serum insulin levels observed in the test sample group. There are reports that UCP2 expression is induced in the liver upon administration of oleic acid or a PPARα agonist, and that in adipose tissues its expression is also induced by PPARγ.

### Test Example 2

### 1. Subjects

Four healthy males were used as subjects and examined for their energy metabolism measurements and for combined use of a sample with breakfast, using the same test sample and control as used in Test Example 1 (commercially-available oral or tube nutritional agent: Table 8. Every 100 g contained 9.8 g of dextrin and 3.9 g of sucrose as carbohydrates, and 3.3 g of vegetable oil as fat). Table 9 shows the physical findings and blood biochemical data of the subjects. A 48-year old female IGT patient was examined after long-term combined use of the test sample with breakfast, after explaining the substance of this study and obtaining informed consent. The physical findings and blood biochemical data of the subject were: BMI: 31.8 kg/m²; body weight: 72.6 kg; fasting blood glucose: 115 mg/dl; HbA1c 5.2%; total cholesterol: 229 mg/dl; triacylglycerol 97 mg/dl; and HDL cholesterol: 59 mg/dl.

TABLE 8

| | In 250 mL (250 kcal) | |
|---|---|---|
| Protein | 8.8 | g |
| Fat | 8.8 | g |
| Carbohydrate | 34.3 | g |
| Vitamin A | 625 | IU |
| Vitamin D | 50 | IU |
| Vitamin E | 7.5 | mg |
| Vitamin K | 17.5 | μg |
| Vitamin C | 38 | mg |
| Vitamin B₁ | 0.38 | mg |
| Vitamin B₂ | 0.43 | mg |
| Vitamin B₆ | 0.50 | mg |
| Vitamin B₁₂ | 1.5 | μg |
| Choline | 0.13 | g |
| Folic acid | 50 | μg |
| Niacin | 5.0 | mg |
| Pantothenic acid | 1.25 | mg |
| Biotin | 38 | μg |
| Sodium | 0.20 | g |
| Potassium | 0.37 | g |
| Chlorine | 0.34 | g |
| Calcium | 0.13 | g |
| Phosphorus | 0.13 | g |
| Magnesium | 50 | mg |
| Manganese | 0.50 | mg |
| Copper | 0.25 | mg |
| Zinc | 3.75 | mg |
| Iron | 2.25 | mg |

TABLE 9

| (n = 4) | Range | Mean ± Standard Error |
|---|---|---|
| Age (years old) | 30-33 | 31.8 ± 0.6 |
| BMI (kg/m²) | 22.7-27.1 | 24.0 ± 1.1 |
| Cholesterol (mg/dl) | 156-229 | 188.8 ± 15.1 |
| Triacylglycerol (mg/dl) | 107-137 | 122.5 ± 6.5 |
| HDL cholesterol (mg/dl) | 41.4-57.5 | 51.2 ± 3.5 |
| Fasting blood glucose (mg/dl) | 93-98 | 95.3 ± 1.1 |

### 2. Method

1) Measurement of Metabolism after Intake of the Test Sample and Control

Experiments were carried out using crossover tests in which subjects took the test sample or the control on two different days. In the early morning fasting state the subjects were rested for 30 minutes on a reclined bed, and their resting metabolism was measured by respiratory gas analysis using an indirect calorimeter (Minato Medical Science). After measuring their resting metabolism, the subjects ingested 250 kcal of the test sample or control and their metabolism was measured after 30, 60, 90, 120, 150, and 180 minutes of ingestion. Measurements at each time sampled breath for 15 minutes, excluded the first five minutes from the data as the time until stabilization, then averaged the measurements for the remaining ten minutes and used this as data.

2) Examination of the Test Sample and Control when Used in Combination with an Ordinary Diet

Experiments were carried out using crossover tests in which the subjects take the test diet and a test sample (test sample-loaded group), or the test diet and a control (control-loaded group) for breakfast. The total energy of the breakfast was 517 kcal, and each group ingested 250 kcal of test sample or control, which is approximately half the total energy of the breakfast. Blood was collected at fasting early in the morning, and this was defined as blood collected at zero minutes after breakfast. Blood collection was followed by intake of breakfast, and blood was collected 15, 30, 60, and 120 minutes after starting breakfast. After blood collection at 120 minutes, the subjects were allowed to move freely for three hours until lunch. Blood was collected before starting lunch, and 30, 60, and 120 minutes after starting lunch. For lunch, both loaded groups took meals with the same content. The composition of the breakfasts and lunches is shown in Table 10. Plasma glucose (PG), serum insulin (IRI), and serum free fatty acids (FFA) were measured using the collected blood. Blood biochemical examination was also performed on the blood collected at fasting early in the morning.

TABLE 10

| Breakfast | Test diet + test sample | Test diet + control |
|---|---|---|
| Energy (kcal) | 267 + 250 | 267 + 250 |
| Carbohydrate (%) | 55.6 | 57.7 |
| Protein (%) | 17.2 | 14.3 |
| Fat (%) | 27.2 | 28.0 |

| Lunch | Test diet |
|---|---|
| Energy (kcal) | 748 |
| Carbohydrate (%) | 63.1 |
| Protein (%) | 14.1 |
| Fat (%) | 22.8 |

### 3) Examination of Long-Term Test Sample Administration

This study was performed following approval by the Tokushima University Hospital Ethics Committee. The test sample was administered for three months to an impaired glucose tolerance (IGT) patient, who was then examined. The daily energy intake, which continued the diet therapy carried out until that stage, was standard body weight×35 kcal/day (1,800 kcal). For three months the subject took meals in which 250 kcal of daily breakfast was substituted with 250 kcal of the test sample. Blood was collected before starting the long-term administration examination, and 45 and 90 days after starting the examination, and fasting PG and hemoglobin A1c (HbA1c) were measured. Body weight and body fat percentage were also measured at the time of blood collection using a body fat scale.

### 3. Statistical Treatment

The results are indicated as mean±standard error (mean±SE), and significant difference tests were carried out using paired t-tests.

### <Results>

1) Comparison of Changes Over Time in Respiratory Quotient (RQ) and in the Amount of Carbohydrate and Fat Burned Up to 180 Minutes after Ingestion of the Test Sample and Control

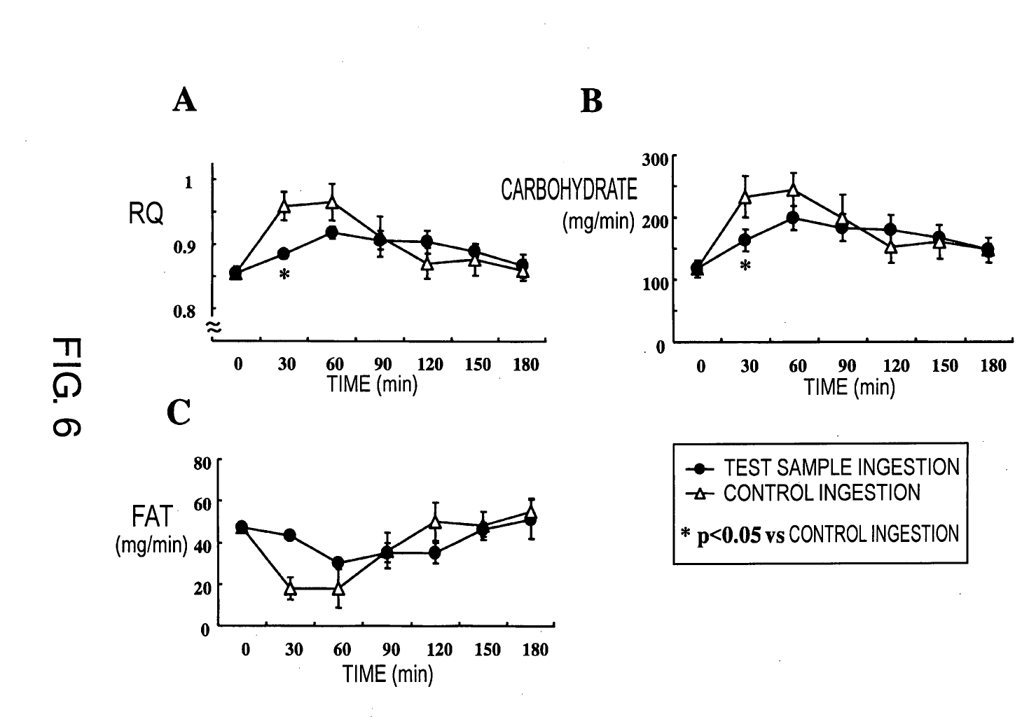

FIG. 6 shows changes over time in RQ and in the amount of carbohydrate and fat burned up to 180 minutes after ingestion of the test sample and control. RQ after ingestion of the test sample increased more slowly than after ingestion of the control, showing a significantly lower value at 30 minutes after ingestion (P<0.05), as well as a lower maximum value. The maximum values for both the test sample and the control occurred at 60 minutes, and were 0.919±0.009 and 0.966±0.028, respectively. When the test sample was ingested, RQ reached a maximum value and then remained nearly constant, whereas in the case of the control, RQ dropped rapidly from its maximum value. The post-ingestion increase in the amount of carbohydrate burned was less for the test sample than for the control, and was continuously maintained at an average of around 170 mg/min, showing little change from the amount burned at fasting. On the other hand, when the control was ingested, the amount of carbohydrate burned rapidly increased to more than 240 mg/min, and then rapidly decreased. Thirty minutes after ingestion of the test sample, significantly less carbohydrate had been burned than was the case for the control (p<0.05). The amount of fat burned after test sample ingestion varied less from the fasting amount than did the control, and was constantly maintained at a high level of about 40 mg/min. Thirty minutes after control ingestion the amount of burned fat decreased to less than 20 mg/min, and from 90 minutes was almost the same as when the test sample was ingested.

### 2) Comparison of Changes in PG, IRI, and FFA Over Time in the Test Sample-Loaded Group and Control-Loaded Group

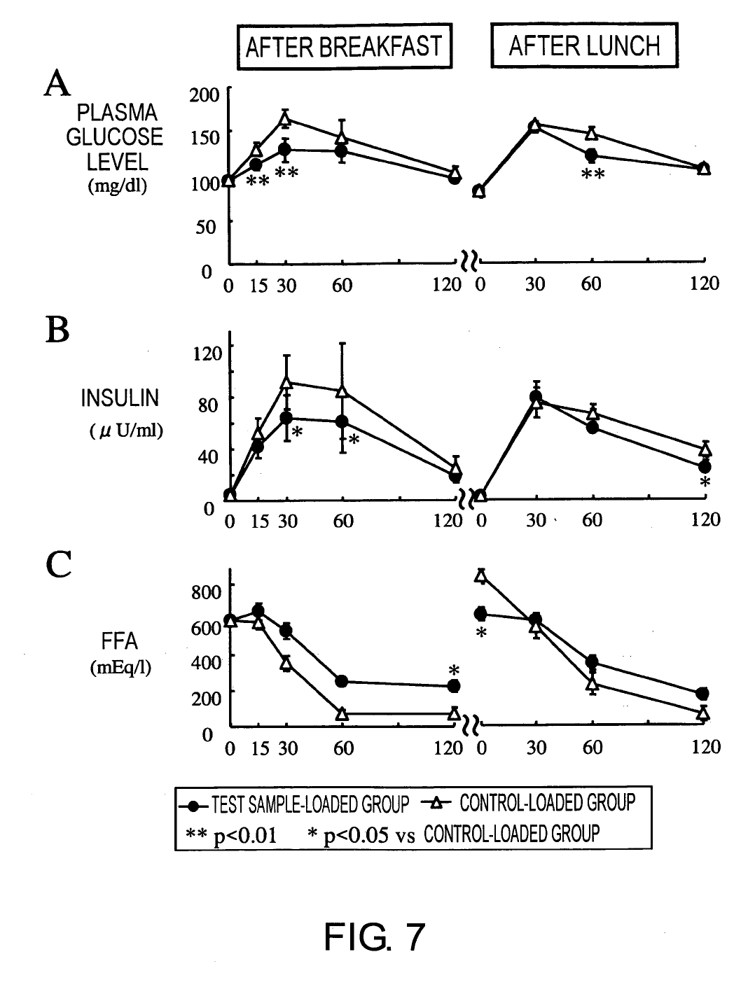

FIG. 7A shows the plasma glucose level curve. PG after ingesting breakfast reached a maximum value 30 minutes after ingestion for both the test sample-loaded group and the control-loaded group, and at 120 minutes this value had almost returned to fasting level. In the test sample-loaded group, the values at 15 and 30 minutes were significantly (p<0.01) less than in the control-loaded group. The values for the test sample-loaded group and the control-loaded group were 112.7±5.6 mg/dl and 130.0±7.5 mg/dl respectively at 15 minutes (p<0.01), and 129.0±12.7 mg/dl and 164.7±9.7 mg/dl (p<0.01) respectively at 30 minutes. PG after lunch reached a maximum value at 30 minutes for both the test sample-loaded group and the control-loaded group, and showed nearly the same 30-minute values of 165.0±6.3 mg/dl and 169.0±4.2 mg/dl respectively. However, the test sample-loaded group dropped from the maximum more rapidly, showing a significantly lower 60-minute value then the control-loaded group. The values at 60 minutes were 130.3±8.0 mg/dl and 158.0±7.8 mg/dl (p<0.01) respectively.

FIG. 7B shows the IRI curve. As for PQ post-breakfast IRI reached a maximum value 30 minutes after ingestion in both loaded groups. The values at 30 and 60 minutes were significantly (p<0.05) lower in the test sample-loaded group than in the control-loaded group. The values for the test sample-loaded group and the control-loaded group were 64.1±17.8 μU/mL and 91.7±20.9 μU/mL respectively at 30 minutes (p<0.05), and 61.0±24.3 μU/mL and 84.8±36.9 μU/mL respectively at 60 minutes (p<0.05). As for PG, post-lunch IRI reached a maximum value 30 minutes after ingestion in both loaded groups (77.9±11.9 μU/mL and 73.9±11.5 μU/mL (p<0.05)). However, the test sample-loaded group dropped from the maximum more rapidly and showed a significantly lower 120-minute value than the control-loaded group. The values at 120 minutes were 23.8±5.7 μU/mL and 37.5±5.6 μU/mL (p<0.05), respectively.

The FFA curve is shown in FIG. 7C. After ingesting breakfast, the FFA of the test sample-loaded group decreased more slowly and showed a significantly higher value at 120 minutes than the control-loaded group. The 120-minute values for the test sample-loaded group and the control-loaded group were 226±30 mEq/L and 75±33 mEq/L (p<0.05) respectively. In addition, at zero minutes after lunch the test sample-loaded group showed a significantly lower value than the control-loaded group (628±36 mEq/L and 848±27 mEq/L (p<0.05)).

3) Comparison Between the Test Sample-Loaded Group and the Control-Loaded Group of Areas Under the Curves (AUC) of PG and IRI Up to 120 Minutes after Ingesting Breakfast and Lunch

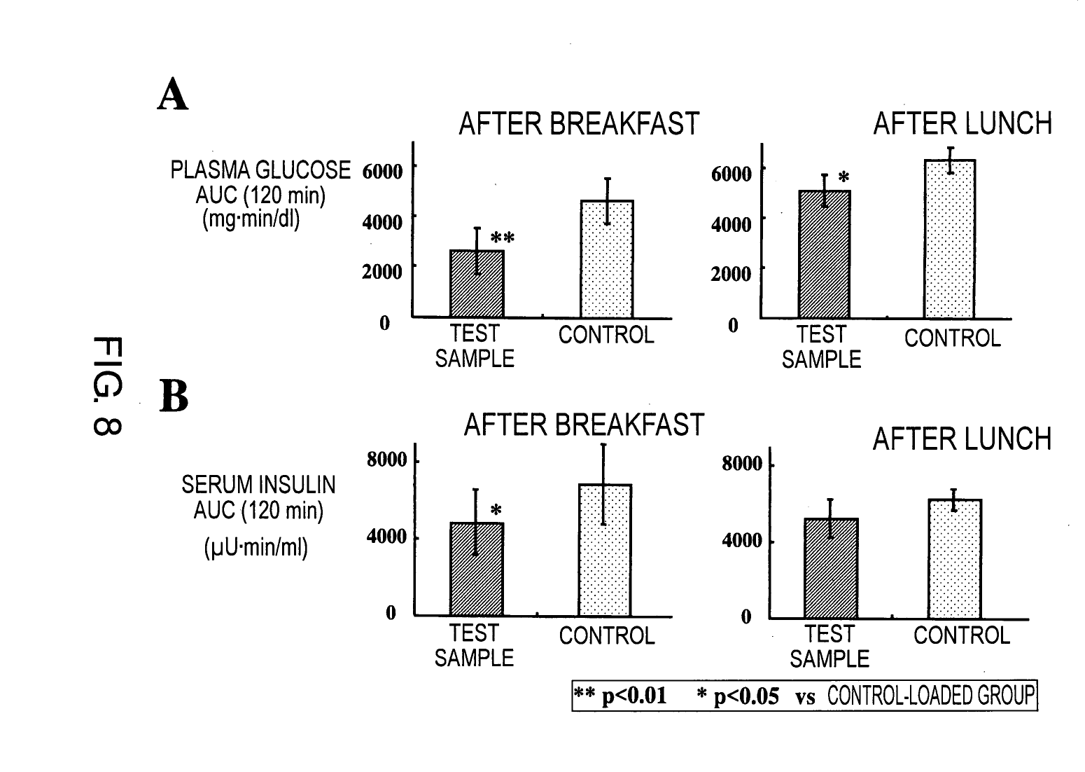

The areas under the plasma glucose curves up to 120 minutes (AUC (0-120 min)) after ingestion of breakfast and lunch are shown in FIG. 8A. The AUC (120 min) after ingesting breakfast in the test sample-loaded group and in the control-loaded group were 2611.0±914.7 mg·min/dl and 4640.0±900.0 mg·min/dl respectively; the test sample-loaded group showed a value significantly (p<0.01) lower by approximately 45% as compared to the control-loaded group. Further, the AUC (120 min) after ingesting lunch in the test sample-loaded group and in the control-loaded group were 5010±629.6 mg·min/dl and 6236±500.3 mg·min/dl respectively; the test sample-loaded group showed a value significantly (p<0.05) lower by approximately 20% as compared to the control-loaded group.

The serum insulin AUC (0-120 min) up to 120 minutes after ingestion of breakfast and lunch are shown in FIG. 8B. AUC (120 min) after ingesting breakfast in the test sample-loaded group and in the control-loaded group were 4847.3±1685.4 mg·min/dl and 6849.5±2083.3 mg·min/dl respectively; the test sample-loaded group showed a value significantly (p<0.05) lower by approximately 30% as compared to the control-loaded group. AUC (120 min) after ingesting lunch in the test sample-loaded group and in the control-loaded group were 5244.0±997.6 mg·min/dl and 6240.0±566.8 mg·min/dl respectively; a significant difference could not be observed, however, the values in the test sample-loaded group showed a tendency to be lower than those in the control-loaded group.

### 4) Effects of Long-Term Administration of the Test Sample

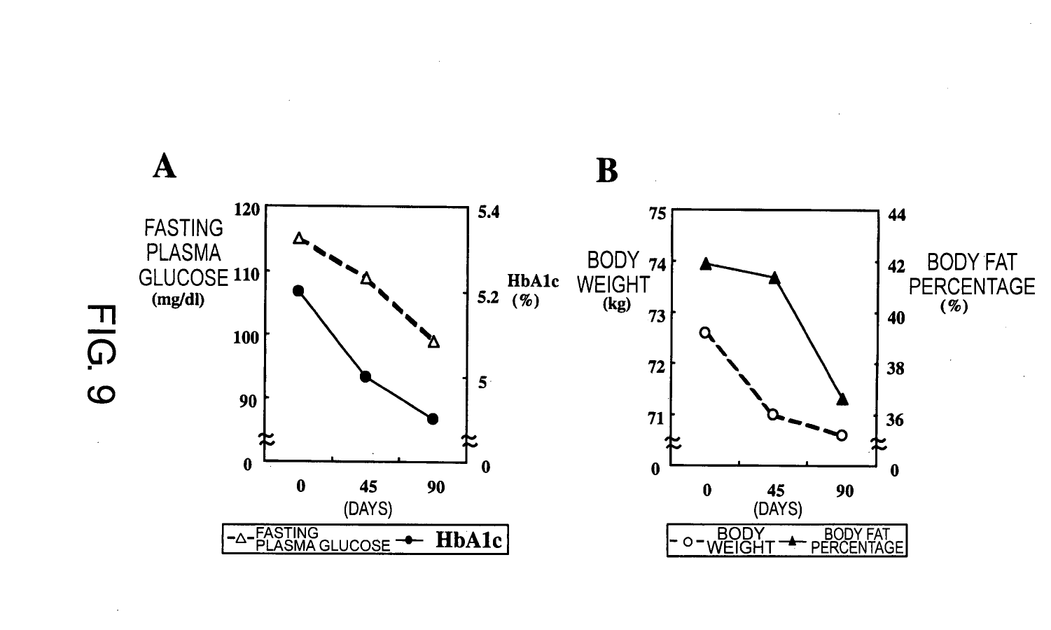

FIG. 9A shows the change in fasting plasma glucose and HbA1c in an IGT patient. When the test substance was administered at breakfast for three months, the fasting blood glucose of the IGT patient decreased from the initial value of 115 mg/dl to 99 mg/dl 90 days later, and HbA1c decreased from 5.2% to 4.9%. The changes in body weight and body fat percentage are shown in FIG. 9B. In three months, body weight decreased from 72.6 kg to 70.6 kg and body fat percentage decreased from 41.9% to 36.6%. No change was observed in blood lipids.

### INDUSTRIAL APPLICABILITY

The nutritional compositions of the present invention have the activity of enhancing the production of PPARs and adiponectin, and are useful as oral and enteral nutritional agents.

1. A nutritional composition for enhancing the production of a PPAR and/or PPAR-associated factor, wherein the composition comprises proteins, fats, and carbohydrates such that proteins account for 10% to 25% of its energy, fats account for 10% to 35% of its energy, and carbohydrates account for 40% to 60% of its energy, and wherein oleate esters account for 60% to 90% of the energy in the fats, and palatinose and/or trehalose account for 60% to 100% of the energy in the carbohydrates.

2. The composition of claim 1, wherein the PPAR is a PPARα and/or a PPARγ.

3. The composition of claim 1, wherein the PPAR is a PPARγ.

4. The composition of claim 1, wherein the PPAR-associated factor is adiponectin.

5. The composition of claim 1, which comprises at least one fat selected from a milk phospholipid, a soybean lecithin, a high oleic sunflower oil, and a perilla oil.

6. A food for treating a patient with diabetes, treating a patient with impaired glucose tolerance, or preventing obesity, which comprises a nutritional composition for enhancing the production of a PPAR and/or PPAR-associated factor, wherein the composition comprises proteins, fats, and carbohydrates such that proteins account for 10% to 25% of its energy, fats account for 10% to 35% of its energy, and carbohydrates account for 40% to 60% of its energy, and wherein oleate esters account for 60% to 90% of the energy in the fats, and palatinose and/or trehalose account for 60% to 100% of the energy in the carbohydrates.

7. An insulin sensitivity-restoring and/or body fat burning composition, comprising a nutritional composition for enhancing the production of a PPAR and/or PPAR-associated factor, wherein the composition comprises proteins, fats, and carbohydrates such that proteins account for 10% to 25% of its energy, fats account for 10% to 35% of its energy, and carbohydrates account for 40% to 60% of its energy, and wherein oleate esters account for 60% to 90% of the energy in the fats, and palatinose and/or trehalose account for 60% to 100% of the energy in the carbohydrates.

8. Use of a nutritional composition for enhancing the production of a PPAR and/or PPAR-associated factor, wherein the composition comprises proteins, fats, and carbohydrates such that proteins account for 10% to 25% of its energy, fats account for 10% to 35% of its energy, and carbohydrates account for 40% to 60% of its energy, and wherein oleate esters account for 60% to 90% of the energy in the fats, and palatinose and/or trehalose account for 60% to 100% of the energy in the carbohydrates in the preparation of an insulin sensitivity-restoring and/or body fat burning composition.

9. A method for restoring insulin sensitivity, which comprises the step of administering to a patient in need of such treatment, a nutritional composition for enhancing the production of a PPAR and/or PPAR-associated factor, wherein the composition comprises proteins, fats, and carbohydrates such that proteins account for 10% to 25% of its energy, fats account for 10% to 35% of its energy, and carbohydrates account for 40% to 60% of its energy, and wherein oleate esters account for 60% to 90% of the energy in the fats, and palatinose and/or trehalose account for 60% to 100% of the energy in the carbohydrates

10. A method for burning body fat, which comprises the step of administering to a patient in need of such treatment, a nutritional composition for enhancing the production of a PPAR and/or PPAR-associated factor, wherein the composition comprises proteins, fats, and carbohydrates such that proteins account for 10% to 25% of its energy, fats account for 10% to 35% of its energy, and carbohydrates account for 40% to 60% of its energy, and wherein oleate esters account for 60% to 90% of the energy in the fats, and palatinose and/or trehalose account for 60% to 100% of the energy in the carbohydrates.
